(19) United States
(12) Patent Application Publication
(10) Pub. No.: US 2025/0345065 A1
(43) Pub. Date: Nov. 13, 2025

(54) Left Atrial Appendage Occluder with Pulmonary Ridge Disc Coverage

(71) Applicant: St. Jude Medical, Cardiology Division, Inc., St. Paul, MN (US)

(72) Inventors: Gary Erzberger, Corcoran, MN (US); Sadie Cronin, Plymouth, MN (US); Kristen T. Morin, St. Paul, MN (US); Michael P. Meyer, Minnetrista, MN (US); Brian Joseph Perszyk, Shoreview, MN (US)

(73) Assignee: St. Jude Medical, Cardiology Division, Inc., St. Paul, MN (US)

(21) Appl. No.: 19/181,424

(22) Filed: Apr. 17, 2025

Related U.S. Application Data
(60) Provisional application No. 63/645,906, filed on May 12, 2024.

(51) Int. Cl. A61B 17/12; A61B 17/00; A61B 90/00

(52) U.S. Cl. CPC A61B 17/12122; A61B 17/12172; A61B 90/39; A61B 2017/00477; A61B 2090/3966

(57) ABSTRACT

According to one aspect of the disclosure, a collapsible and expandable medical device for occluding a left atrial appendage (“LAA”), includes a proximal disc, a distal lobe, and a connecting member. The proximal disc may be configured to cover an ostium of the LAA in an implanted condition of the medical device. The distal lobe may be configured to be received within a cavity of the LAA in the implanted condition of the medical device. The distal lobe may include a central longitudinal axis extending therethrough in an expanded condition of the medical device. The connecting member may connect the proximal disc to the distal lobe. In the expanded condition of the medical device, the proximal disc may not be radially symmetric about the central longitudinal axis of the distal lobe.

### CROSS-REFERENCE TO RELATED APPLICATIONS

This application claims priority to the filing date of U.S. Provisional Patent Application No. 63/645,906, filed May 12, 2024, the disclosure of which is hereby incorporated by reference herein.

### FIELD OF THE DISCLOSURE

The present disclosure relates generally to medical devices that are used in the human body. In particular, the present disclosure is directed to an occlusion device having a configuration that allows for more consistent and stable anchoring and sealing of the occlusion device within a tissue cavity. More specifically, the present disclosure is directed to an occlusion device with features to reduce pockets or other areas of stagnation post-implantation to reduce the risk of thrombus formation and stroke risk.

### BACKGROUND

An occluder is a medical device used to treat (e.g., occlude) tissue at a target site within the human body, such as an abnormality, a vessel, an organ, an opening, a chamber, a channel, a hole, a cavity, a lumen, or the like. For example, an occluder may be used for Left Atrial Appendage (“LAA”) closures. An LAA is a normal anatomical structure in which there is a sac in the muscle wall of the left atrium. When a patient experiences atrial fibrillation (“AFib”), a blood clot may be formed within the LAA which may become dislodged and enter into the blood stream. By occluding the LAA, the release of blood clots from the LAA may be significantly reduced, if not eliminated. Various techniques have been developed to occlude the LAA. For instance, balloon-like devices have been developed that are configured to be implanted completely within the cavity of the LAA, while surgical techniques have also been developed where the cavity of the LAA is inverted and surgically closed.

Despite these techniques, it would be advantageous to provide an improved occlusion device that offers a reduced risk of adverse events such as thrombus formation and stroke resulting from embolization of the thrombus.

### SUMMARY OF THE DISCLOSURE

According to one aspect of the disclosure, a collapsible and expandable medical device for occluding a left atrial appendage (“LAA”) includes a proximal disc configured to cover an ostium of the LAA in an implanted condition of the medical device. The device may include a distal lobe configured to be received within a cavity of the LAA in the implanted condition of the medical device, and the distal lobe may include a central longitudinal axis extending therethrough in an expanded condition of the medical device. A connecting member may connect the proximal disc to the distal lobe. In the expanded condition of the medical device, the proximal disc may not be radially symmetric about the central longitudinal axis of the distal lobe. In the expanded condition of the medical device, the connecting member may extend along a central longitudinal axis that is oblique to the central longitudinal axis of the distal lobe. In the expanded condition of the medical device, the proximal disc may extend along a central longitudinal axis, and the central longitudinal axis of the proximal disc may be offset and parallel to the central longitudinal axis of the distal lobe. The distal lobe may be radially symmetric about the central longitudinal axis of the distal lobe, and the proximal disc may be radially symmetric about the longitudinal axis of the proximal disc. In the expanded condition of the medical device, the central longitudinal axis of the proximal disc may pass through the distal lobe. At least one radiopaque marker may be coupled to a radially outer surface of the distal lobe at a position of the radially outer surface of the distal lobe having a minimum spaced distance radially from the central longitudinal axis of the proximal disc. At least one radiopaque marker may be coupled to a radially outer surface of the proximal disc at a position of the radially outer surface of the proximal disc having a maximum spaced distance radially from the central longitudinal axis of the distal lobe. At least one radiopaque marker may be coupled to a radially outer surface of the distal lobe at a position of the radially outer surface of the distal lobe having a maximum spaced distance radially from the central longitudinal axis of the proximal disc. In the expanded condition of the medical device, the proximal disc may extend along a central longitudinal axis, and the proximal disc may not be radially symmetric about the central longitudinal axis of the proximal disc. In the expanded condition of the medical device, the proximal disc may include an outer periphery with a first portion and a second portion that together form the outer periphery, the first portion having a first radius of curvature, the second portion having a second radius of curvature larger than the first radius of curvature. The first portion of the outer periphery of the proximal disc may form an arc having a center that is substantially coaxial with the central longitudinal axis of the distal lobe. The second portion of the outer periphery of the proximal disc may form an arc having a center that is not coaxial with the central longitudinal axis of the distal lobe.

According to another aspect of the disclosure. a collapsible and expandable medical device for occluding a left atrial appendage may include a proximal disc configured to cover an ostium of the LAA in an implanted condition of the medical device. A distal lobe may be configured to be received within a cavity of the LAA in the implanted condition of the medical device. The distal lobe may include a central longitudinal axis extending therethrough in an expanded condition of the medical device. A connecting member may connect the proximal disc to the distal lobe. The proximal disc may include a radially outer zone forming an outer periphery of the proximal disc and a radially inner zone that is positioned radially inwards of the radially outer zone, the radially inner zone having a stiffness that is greater than a stiffness of the radially outer zone. The proximal disc may be formed of one or more strands of wires braided together, and the one or more strands may be located in the radially outer zone and may have a thickness that is smaller than a thickness of one or more of the strands located in the radially inner zone. The proximal disc may be formed of one or more strands of wires braided together into a braided fabric with each braid wire crossing forming a pick, a pick rate being defined as the number of picks per inch of braided fabric, and the radially outer zone may have a pick rate that is smaller than a pick rate of the radially inner zone. The radially inner zone of the proximal disc may be formed of one or more strands of wires braided together, and the radially outer zone of the proximal disc may include a polymer. The radially outer zone of the proximal disc may be devoid of wire strands. The radially outer zone of the proximal disc may include at least one wire strand. The at least one wire strand in the radially outer zone of the proximal disc may undulate between a radially outer edge of the radially inner zone and a radially outer edge of the radially outer zone in a circumferential direction of the radially outer zone. The proximal disc may be formed by one or more wire strands that form a plurality of spindles extending from a central portion of the proximal disc toward a radially outer edge of the proximal disc such that the radially inner zone has a density of wire strands that is greater than a density of wire strands in the radially outer zone. The proximal disc may be formed by one or more wire strands that form a plurality of wire loops. A first group of the wire loops may be positioned relatively close to a radial center of the proximal disc and may each have a first size, a second group of wire loops may be positioned relatively close to an outer peripheral edge of the proximal disc and may each have a second size, and the first size may be smaller than the second size such that the radially inner zone has a density of wire strands that is greater than a density of wire strands in the radially outer zone.

### BRIEF DESCRIPTION OF THE DRAWINGS

FIG. 1 illustrates a known medical device.

FIG. 2 is a schematic diagram of a delivery system in accordance with the present disclosure.

FIG. 3 is a highly schematic view of the medical device of FIG. 1 implanted within an exemplary LAA.

FIGS. 4A-D are various views of another embodiment of an occluder with similarities to the medical device of FIG. 1.

FIGS. 5A-B are side and bottom views, respectively, of another embodiment of an occluder with similarities to the medical device of FIG. 1.

FIG. 6 is a highly schematic view of a version of the medical device of FIG. 1 implanted within an exemplary LAA.

FIG. 7A is a highly schematic view of another embodiment of an occluder with similarities to the medical device of FIG. 1 implanted in an exemplary LAA.

FIGS. 7B-F are examples of alternative embodiments of a disc of an occluder similar to that shown in FIG. 7A, having an outer area with relatively high conformability compared to an inner area.

FIG. 7G is an example of an alternative embodiment of the occluder of FIG. 7A with a double braid construction.

### DETAILED DESCRIPTION OF THE DISCLOSURE

The present disclosure relates generally to medical devices that are used in the human body. Specifically, the present disclosure provides medical devices including occlusion devices having features for enhancing engagement and sealing of the occluder within the tissue in which it is implanted, while minimizing risks of thrombus formation and risks of resulting stroke. The disclosed embodiments may lead to more consistent and improved patient outcomes. It is contemplated, however, that the described features and methods of the present disclosure as described herein may be incorporated into any number of systems as would be appreciated by one of ordinary skill in the art based on the disclosure herein.

Although the exemplary embodiment of the medical device is described as treating a target site including a LAA, it is understood that the use of the term “target site” is not meant to be limiting, as the medical device may be configured to treat any target site, such as an abnormality, a vessel, an organ, an opening, a chamber, a channel, a hole, a cavity, or the like, located anywhere in the body. The term “vascular abnormality,” as used herein is not meant to be limiting, as the medical device may be configured to bridge or otherwise support a variety of vascular abnormalities. For example, the vascular abnormality could be any abnormality that affects the shape of the native lumen, such as an atrial septal defect, a lesion, a vessel dissection, or a tumor. Embodiments of the medical device may be useful, for example, for occluding a patent foramen ovalis (“PFO”), atrial septal defect (“ASD”), ventricular septal defect (“VSD”), or patent ductus arteriosus (“PDA”), as noted above. Furthermore, the term “lumen” is also not meant to be limiting, as the vascular abnormality may reside in a variety of locations within the vasculature, such as a vessel, an artery, a vein, a passageway, an organ, a cavity, or the like. As used herein, the term “proximal” refers to a part of the medical device or the delivery device that is closest to the operator, and the term “distal” refers to a part of the medical device or the delivery device that is farther from the operator at any given time as the medical device is being delivered through the delivery device. In addition, the terms “deployed” and “implanted” may be used interchangeably herein.

Some embodiments of the present disclosure provide an improved percutaneous catheter directed intravascular occlusion device for use in the vasculature in patients' bodies, such as blood vessels, channels, lumens, a hole through tissue, cavities, and the like, such as a LAA. Other physiologic conditions in the body occur where it is also desirous to occlude a vessel or other passageway to prevent blood flow into or therethrough. These device embodiments may be used anywhere in the vasculature where the anatomical conditions are appropriate for the design.

The medical device may include one or more layers of occlusive material, wherein each layer may be comprised of any material that is configured to substantially preclude or occlude the flow of blood so as to facilitate thrombosis. As used herein, “substantially preclude or occlude flow” shall mean, functionally, that blood flow may occur for a short time, but that the body's clotting mechanism or protein or other body deposits on the occlusive material results in occlusion or flow stoppage after this initial time period.

Some embodiments of the present disclosure may be formed by a plurality of wire strands having a predetermined relative orientation with respect to one another. However, it is understood that according to additional embodiments of the present disclosure, that the medical device could be etched or laser cut from a tube, or the device could comprise an occlusion material coupled to a scaffolding structure or a plurality of slices of a tubular member coupled together.

The present disclosure now will be described more fully hereinafter with reference to the accompanying drawings, in which some, but not all embodiments of the disclosure are shown. Indeed, this disclosure may be embodied in many different forms and should not be construed as limited to the embodiments set forth herein; rather, these embodiments are provided so that this disclosure will satisfy applicable legal requirements. Like numbers refer to like elements throughout.

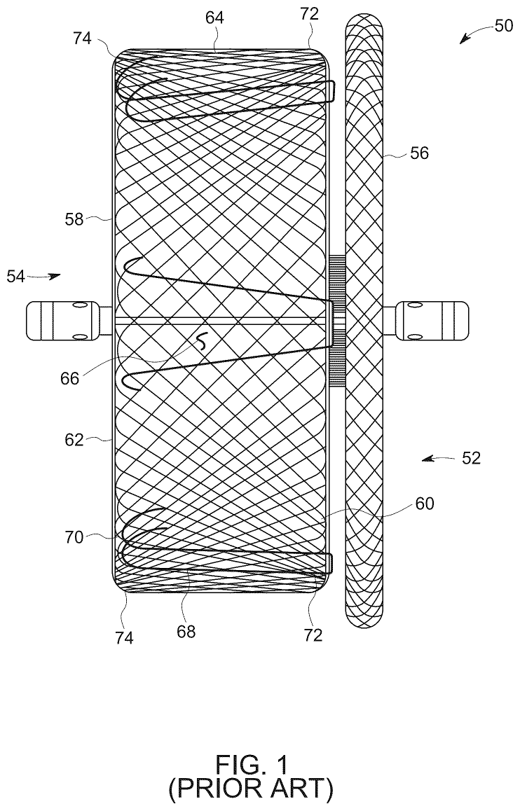

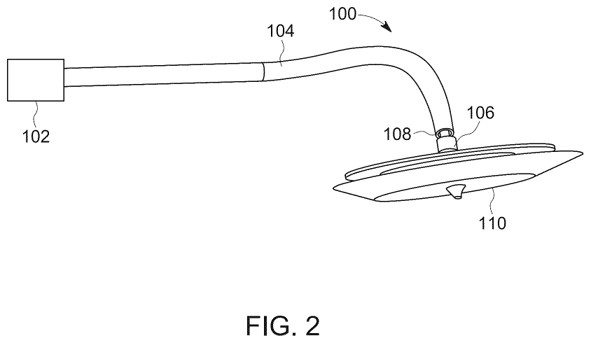

In at least some conventional or known medical devices used for the occlusion of abnormalities, such as a medical device 50 shown in FIG. 1, medical device 50 includes a proximal end 52 and a distal end 54, with a disc 56 at proximal end 52 and a lobe 58 at distal end 54. The lobe 58 has a proximal edge 60 (also referred to as a proximal face), a distal edge 62 (also referred to as a distal face), and a middle or central portion 64 that defines a cavity 66. The medical device 50 also includes stabilizing wires 68 secured to a radially outer or circumferential surface of middle portion 64. The stabilizing wires 68 terminate in a hook 70 at free ends thereof, and thereby facilitate retention of the medical device 50 at a target site and preventing the medical device 50 from becoming dislodged from the target site after deployment.

In this known medical device 50, proximal edge 60 and distal edge 62 adjoin middle portion 64 at a first relatively blunt or sharp (e.g., non-rounded) transition 72 and a second blunt transition 74, respectively. First blunt transition 72 connects proximal edge 60 to middle portion 64 by an approximately 90 degree angle. Likewise, second blunt transition 74 connects distal edge 62 to middle portion 64 by an approximately 90 degree angle. First blunt transition 72 and second blunt transition 74 partially define a generally rectangular cross section to lobe 58, leading to relatively blunt circumferential edges of the device and relatively high radial force applied to the surrounding tissue.

Turning now to FIG. 2, a schematic diagram of a delivery system 100 is shown. Delivery system 100 includes a delivery device 102 including a catheter 104 and a coupling member 106 configured to couple a distal end of a delivery cable 108 to a medical device 110 (which may be any of the occluders described herein) for facilitating the deployment of medical device 110 at a target site. Medical device 110 is deployed to treat the target site, and, in the example embodiment, is an occlusion device (“occluder”).

Much of the disclosure below relates to modifications that may be made to disc 56 to achieve certain desired results, including for example reduction of thrombus formation on the proximal surface of the disc 56 post-implantation. However, it should be understood that various modifications may be made to the lobe 58 as well without departing from the scope of the invention. For example, the medical devices of the present disclosure may include a rounded lobe instead of a lobe having sharper transitions, for example as described in greater detail in U.S. Patent Application Publication No. 2022/0008050, the disclosure of which is hereby incorporated by reference herein. The use of a more rounded lobe may lead to a more uniform radial compression, reduction in radial force applied to surrounding tissue, and reduction in variability of the hook angle of the stabilizing wires, minimizing potential disadvantages of known medical devices. Further, various modifications may be made to the number, shape, and placement of stabilizing wires 68 without departing from the scope of the invention. Thus, it should be understood that while much of the disclosure below focuses on features of, and variations on, disc 56, those features may be implanted in occluders with varying designs of the lobe 58 and stabilizing wires 68.

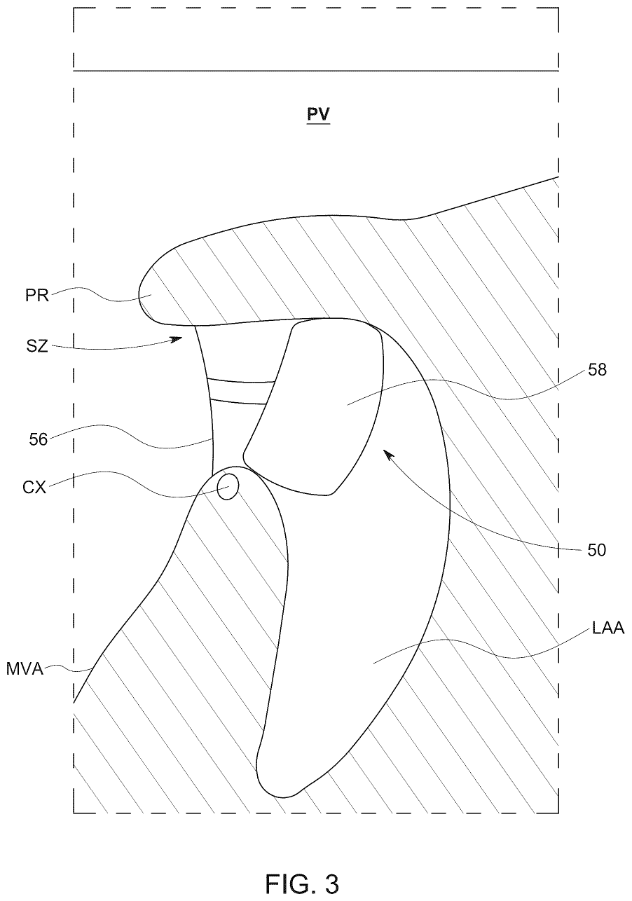

FIG. 3 is a highly schematic view of the occluder 50 of FIG. 1 implanted into a LAA of a patient. During an LAA closure or occlusion procedure, complete sealing of the LAA is an important outcome. In addition to complete sealing, continuous coverage of the ostium of the LAA, through the pulmonary ridge PR has been shown to be a factor in potential thrombus forming on the device. In FIG. 3, locations of the pulmonary vein PV, circumflex artery CX, and mitral valve annulus MVA are also shown and labeled. If the disc 56 of the occluder 50 does not create a smooth transition from the LAA ostium to the pulmonary ridge PR, a blood stagnation zone SZ, which may alternately be referred to as a pocket or triangle, may be formed. This pocket or stagnation zone SZ can be a high-risk area for thrombus to form on the occluder 50, for example on the proximal face of the disc 56. If thrombus forms on the occluder 50 on the proximal surface of the disc 56, the risk for patient stroke may become elevated due to the risk of embolization of the thrombus, and the patient may need to undergo further medication to resolve the thrombus. As should be understood, the creation of the pocket or the stagnation zone SZ shown in FIG. 3 is not desirable.

As described in greater detail below, one or more modifications to occluder 50, with particular emphasis on disc 56, may be made in order to increase the likelihood of the disc 56 to cover the transition area of the patient between the ostium of the LAA and the pulmonary ridge PR to reduce the size of, or eliminate, the pocket or stagnation zone SZ.

One challenge related to modifying the disc 56 to create the smooth transition with the pulmonary ridge PR is that, for many patients, the disc 56 cannot be simply made larger to enhance the transition. For example, if the disc 56 is increased in diameter, although better coverage may be obtained superiorly (e.g., between the ostium of the LAA and the pulmonary ridge PR), the disc 56 would also extend farther inferiorly toward the mitral valve annulus MVA, which can result in interference with the proper functioning of the mitral valve of the patient. As will become clear below, embodiments described herein are generally directed to optimization of one or more features of the occluder 50, particularly the disc 56, to cover the transition between the ostium of the LAA and the pulmonary ridge PR, to reduce or eliminate any pockets or stagnation zones SZ, while also avoiding interference with other surrounding structures, such as the mitral valve annulus MVA.

As noted above, in many patients, simply increasing the diameter of the disc 56 may not be a suitable approach for obtaining better coverage between the ostium of the LAA and the pulmonary ridge PR. However, for some patients, this may be a suitable approach. One example of medical device 50 is the Amplatzer™ Amulet™ left atrial appendage occluder available from Abbott Laboratories. That particular device is offered in eight sizes. Four sizes are considered a relatively small size, which have an axial length of about 7.5 mm for the lobe 58, and four sizes are considered a relatively large size, which have an axial length of about 10 mm for the lobe 58. The “small” sizes may have about a 6 mm differential between the diameter of the lobe 58 and the diameter of the disc 56. For example, the “small” size offerings may include a diameter of the lobe 58 of 16 mm, 18 mm, 20 mm, or 22 mm, with respective diameters of the disc 56 of 22 mm, 24 mm, 26 mm, or 28 mm. The “large” sizes may have about a 7 mm differential between the diameter of the lobe 58 and the diameter of the disc 56. For example, the “large” size offerings may include a diameter of the lobe 58 of 25 mm, 28 mm, 31 mm, or 34 mm, with respective diameters of the disc 56 of 32 mm, 35 mm, 38 mm, or 41 mm. For certain patients with suitable anatomy, medical device 50 may be offered with a diameter of the disc 56 that is between about 8 mm and about 11 mm larger than the diameter of the lobe 58. This increase in ratio of the size of the disc 56 to the size of the lobe 58 may allow for a better transition between the ostium of the LAA and the pulmonary ridge PR so that the pocket or stagnation zone SZ is largely or entirely eliminated, without being so large as to interfere with other structures in the heart such as the mitral valve annulus MVA. In some examples, it may be most desirable to include larger diameter discs for the 22 mm, 25 mm, and 28 mm lobe sizes, although benefit may also be found by including larger diameter discs for the 18 mm, 20 mm, 31 mm and 34 mm lobe sizes. And although a disc size differential of between about 8 mm and about 11 mm is described above, in some embodiments, an additional 3-4 mm for the disc diameter may be beneficial (compared to the typical 6 mm or 7 mm differential described above, e.g., for a total differential of between about 9 mm and about 11 mm) without significantly increased risk of impinging on the mitral valve. It should be understood that the above values are exemplary.

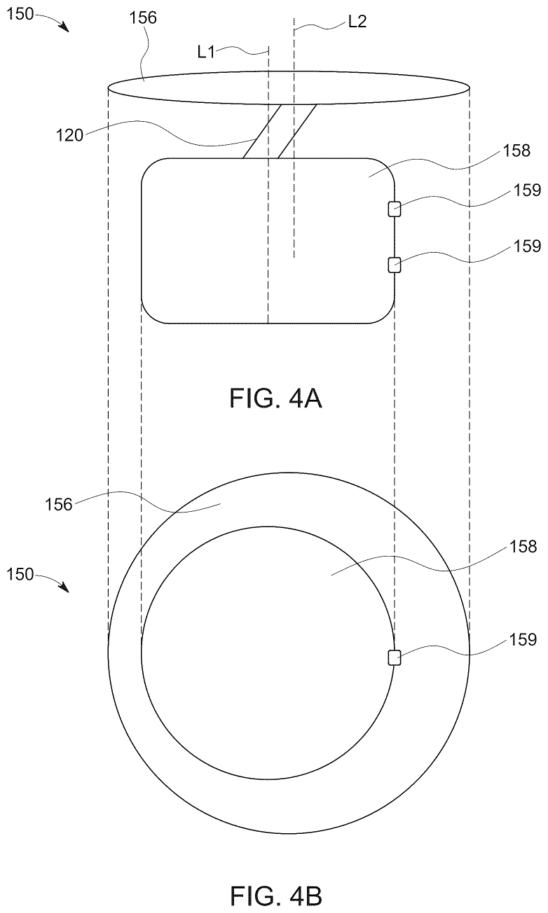

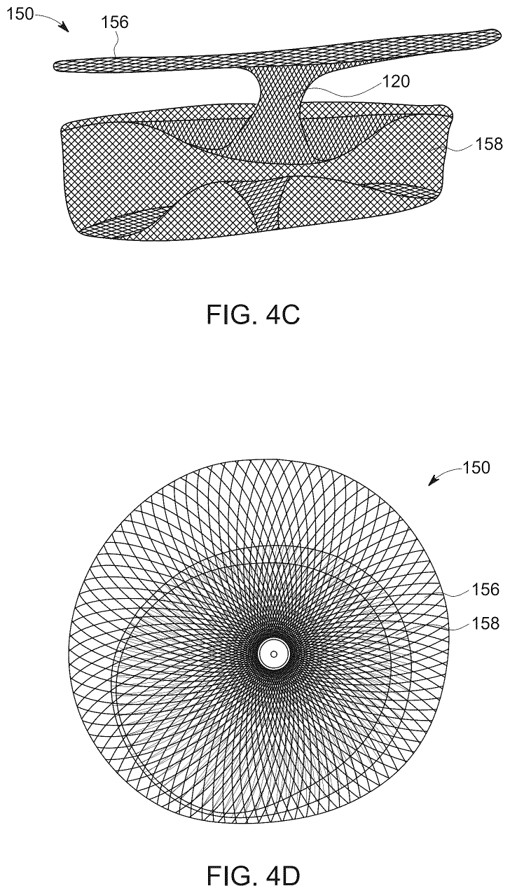

FIGS. 4A-D are various views of another embodiment of an occluder 150 with similarities to the medical device of FIG. 1. It should be understood that, other than the differences described below, occluder 150 may be similar or identical to medical device 50 (and/or medical device 110) described above. A main difference between occluder 150 and medical device 50 is that, in occluder 150, the disc 156 is offset relative to the lobe 158. In other words, whereas disc 56 and lobe 58 (and even the connector or waist portion connecting the two) extend along substantially the same central longitudinal axis, the same is not true for medical device 150. This offset configuration of occluder 150 may be used regardless of the specific differential between the diameter of the disc 156 and the lobe 158 (e.g., whether the disc 156 has a diameter that is 6 mm, 7 mm, 8-11 mm, or another amount larger than the lobe 158). As with medical device 50, the lobe 158 of occluder 150 may be generally cylindrical (e.g. with a circular or substantially circular cross section) and the disc 156 of occluder 150 may similarly be generally cylindrical or disc-shaped (e.g. with a circular or substantially circular cross section). However, a central longitudinal axis L1 passing through a radial center of the lobe 158 is offset from a central longitudinal axis L2 passing through a radial center of the disc 156. As shown next in FIG. 4A, the waist or connecting portion 120 that couples the disc 156 to the lobe 158 may also be generally cylindrical and narrower than the disc 156 and the lobe 158, and each end of the connecting portion 120 may connect to the radial center of the disc 156 or the lobe 158. As a result, the connecting portion 120 in some examples has its own central longitudinal axis (not separately labeled in FIG. 4A) that is oblique to both the central longitudinal axes L1 and L2 of the lobe 158 and the disc 156, respectively. It should be understood that the offset described in connection with occluder 150 is present in the unbiased condition of the occluder 150 (e.g. in the absence of applied forces). It should also be noted that the central longitudinal axes L1 and L2 in some embodiments are substantially parallel to each other, for example because the proximal and distal faces of both the disc 156 and lobe 158 are substantially parallel to each other.

Still referring to FIGS. 4A-D, in the illustrated embodiment, the disc 156 is substantially circular in shape, and as noted above, is shifted or biased laterally relative to the lobe 158. With this configuration, the occluder 150 may be implanted so that the lobe 158 is positioned within the LAA, and in a rotational orientation so that the disc 156 is shifted or biased superiorly (toward the pulmonary ridge PR and away from the mitral valve annulus MVA. By shifting the disc 156 toward the pulmonary ridge PR, the size or diameter of the disc 156 may be increased relative to the lobe 158 (e.g., between 8-11 mm larger than the lobe 158) as there is lower risk of interference with the mitral valve annulus MVA due to the bias of the disc 156. However, as noted above, the disc 156 may have a typical size relative to the lobe 158, such as being 6 mm or 7 mm larger in diameter. In some examples, the increase in the diameter of the disc 156 (compared to the 6 mm or 7 mm differential described above) may be substantially equal to the offset of the disc 156, which may be the distance between the longitudinal axes L1, L2. In some examples, the offset may be between about 3 mm and about 6 mm. In some examples, the offset may be about half the value of the diameter differential between the lobe and the disc. In some examples, the bias or offset may be formed during shape-setting, such as via heat treatment of the occluder 150.

As should be understood, the occluder 150 of FIGS. 4A-D is not rotationally symmetric (even though the lobe 158 and disc 156 may each individually be rotationally symmetric). In other words, because it is desirable that the bias in the disc 156 shifts the disc 156 toward the pulmonary ridge PR (or away from the mitral valve annulus MVA), the lobe 158 must be implanted into the LAA in a particular rotational orientation, or within a suitable range of rotational orientations, in order to position the disc 156 away from the mitral valve annulus MVA. Thus, in some embodiments, one or more indicators, such as radiopaque markers 159 that are visible under fluoroscopy, may be positioned on the lobe 158 in a known rotational position. In the example of FIGS. 4A-B, the radiopaque markers 159 are positioned in alignment with the direction of the offset of the disc 156. With this configuration, the radiopaque markers 159 may be oriented toward the pulmonary ridge PR (or away from the mitral valve annulus MVA) prior to and/or during deployment so that the disc 156 is aligned toward the pulmonary ridge PR. In other embodiments, the markers may be placed in other known rotational positions (e.g., opposite the direction of the bias of the disc 156) to assist with ensuring that the disc 156 is positioned in the desired position upon full deployment. Although markers 159 are described as radiopaque markers, they may be any suitable marker that allows for known positioning, such as echogenic markers or any other suitable indicator. Stated in another way, in the expanded condition of the occluder 150, the longitudinal axis L2 of the disc 156 passes through the lobe 158. In the example of FIGS. 4A-D, the radiopaque markers 159 are positioned on a sidewall or periphery of the lobe 158 at a location that is the minimum distance, in the radial direction, from the longitudinal axis L2 of the disc 156. However, in other embodiments, the radiopaque markers 159 may be positioned on the opposite side of the lobe 158 at a location that is the maximum distance, in the radial direction, from the longitudinal axis L2 of the disc 156. Further, although radiopaque markers 159 are shown and described as being positioned on the lobe 158, it should be understood that radiopaque markers (or other indicators) could instead, or additionally, be positioned on the disc 156 for generally the same reasons. In some examples, providing radiopaque (or other) markers on the disc 156 may allow observation of the orientation of the disc 156 while still collapsed in the delivery sheath prior to full deployment of the occluder 150.

It should be understood that although disc 156 is described and shown as circular, in other embodiments it may have the shape of an ellipse or an oval, in which the major or long axis of the oval is configured to be oriented toward the pulmonary ridge PR upon implantation. In these embodiments, the connecting portion 120 may extend at an oblique angle that is generally aligned with the long axis of the oval disc 156. With this configuration, the additional length of disc 156 provided by the oval shape may help better cover the pulmonary ridge PR upon implantation, but the extra length at the opposite end of the disc 156 would not necessarily risk impinging on the mitral valve due to the connecting portion 120 shifting one of the long ends of the oval disc 156 closer to the central longitudinal axis L1 of the lobe 158.

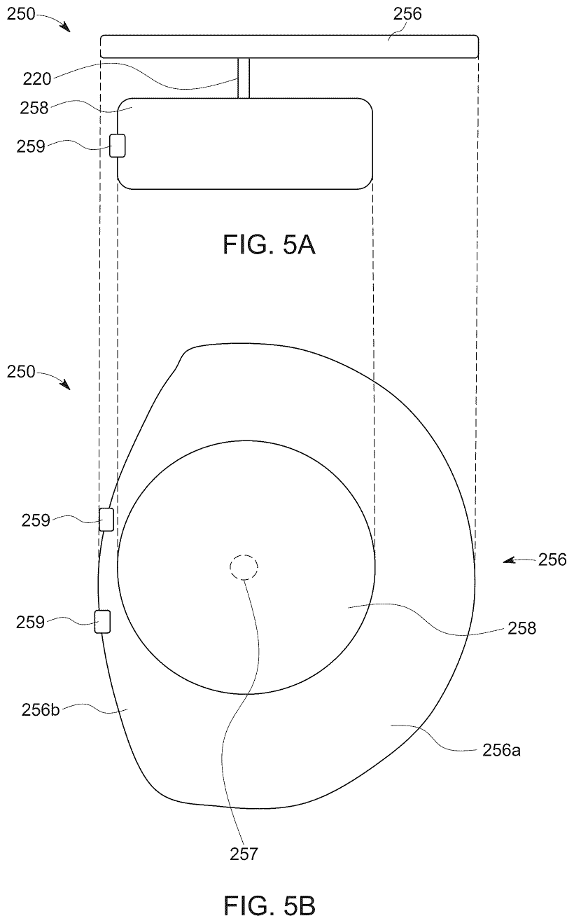

FIGS. 5A-B are side and bottom views, respectively, of another embodiment of an occluder 250 with similarities to the medical device 50 of FIG. 1 and the occluder 150 of FIGS. 4A-D. It should be understood that, other than the differences described below, occluder 250 may be similar or identical to medical device 50 (and/or medical device 110 or occluder 150) described above. A main difference between occluder 250 and medical device 50 is that, in occluder 250, the disc 256 is not circular and is not rotationally symmetric. In other words, while disc 56 and disc 156 are each individually circular and rotationally symmetric, disc 256 is not. It should be understood that, although occluder 150 overall is not rotationally symmetric, the individual components of the disc 156 and lobe 158 are substantially rotationally symmetric. Lobe 258 may be generally cylindrical with a substantially circular cross-section, and the connection portion 220 may be substantially centered along the lobe 258 such that a central longitudinal axis of the connection portion 220 is substantially coaxial with the central longitudinal axis of the lobe 258.

In the example of FIGS. 5A-B, the disc 256 may include a first portion 256a, and a second portion 256b. The first portion 256a may be substantially semi-circular, for example forming an arc with an angle of between about 180 degrees and about 225 degrees, or up to above 270 degrees. In some examples, the arc formed by the first portion 256a may have a center that lies substantially along the central longitudinal axes of the lobe 258 and/or the connection section 220. As best shown in FIG. 5B, a delivery device connector 257 may be positioned on the disc 256 at the center of the arc forming the first portion 256a. The delivery device connector 257 may function to gather ends of the strands forming the braid of the disc 256 and/or to connect to a delivery device (e.g., via internal threading), such as by connecting to the coupling member 106 of delivery device 102. Suitable examples of delivery device connector 257, which may be alternatively described as a hub or end screw, are described in greater detail in U.S. Pat. No. 8,758,389, the disclosure of which is hereby incorporated by reference herein. The second portion 256b of the disc 256 may be smaller than the first portion 256a, and for example may have a larger radius of curvature than the first portion 256a such that the second portion 256b of the disc 256 is flatter than the first portion 256a of the disc 256a. With this configuration, the distance between the delivery device connector 257 and the outer edge of the first portion 256a of the disc 256 is larger than any distance between the delivery device connector 257 and the outer edge of the second portion 256b. Further, while the distance between the delivery device connector 257 and the outer edge of the second portion 256b may vary along the length of the second portion 256b, the minimum distance may be near the center of the outer edge of the second portion 256b, and the maximum distances may be near where the ends of the second portion 256b meet corresponding ends of the first portion 256a. In some examples, the second portion 256b of the disc 256 may be substantially flat. In some examples, the second portion 256b of the disc 256 may be provided with the typical diameters described above for a disc for a particular lobe size, while the first portion 256a of the disc 256a may be provided with the larger disc diameters described above for the particular lobe size.

As should be understood, the occluder 250 of FIGS. 5A-B is not rotationally symmetric because the disc 256 is not rotationally symmetric. In some examples, it is desirable for the center of the arc forming the second portion 256b to be oriented toward the mitral valve annulus MVA and the center of the arc forming the first portion 256a to be oriented toward the pulmonary ridge PR. Because of this, the lobe 258 must be implanted into the LAA in a particular rotational orientation, or within a suitable range of rotational orientations, in order to achieve this positioning of the different portions 256a, 256b of the disc 256. Thus, in some embodiments, one or more indicators, such as radiopaque markers 259 that are visible under fluoroscopy, may be positioned on the lobe 258 (shown in the example of FIG. 5A) and/or on the disc 256 (shown in the example of FIG. 5B) in a known rotational position. In the example of FIGS. 5A-B, the radiopaque markers 259 are positioned in alignment with or adjacent to the center of the arc forming the second portion 256b of the disc 256 (on the lobe 258 in FIG. 5A, on the disc 256 in FIG. 5B). With these configurations, the radiopaque markers 259 may be oriented toward the mitral valve annulus MVA (or away from the pulmonary ridge PR) prior to and/or during deployment so that the smallest radius of the disc 256 is aligned toward the mitral valve annulus MVA and the largest radius of the disc 256 is aligned toward the pulmonary ridge PR. In other embodiments, the markers may be placed in other known rotational positions to assist with ensuring that the disc 256 is positioned in the desired rotational position upon full deployment. Although markers 259 are described as radiopaque markers, they may be any suitable marker that allows for known positioning, such as echogenic markers or any other suitable indicator.

Although the first portion 256a of the disc 256 is generally shown and described as semicircular (or otherwise having a substantially constant radius of curvature), in other embodiments, the first portion 256a of the disc 256 may have the shape of a portion of an oval or a portion of an ellipse, for example including one end of the major or long axis of the oval, with the second end of the major or long axis of the oval being replaced with the second portion 256b of the disc 256. In some configurations, this shape may help provide better coverage of the pulmonary ridge PR if the occluder 250 is implanted with the long axis of the partially oval disc 256 oriented toward the pulmonary ridge PR, with the second relatively flat portion 256b oriented toward the mitral valve.

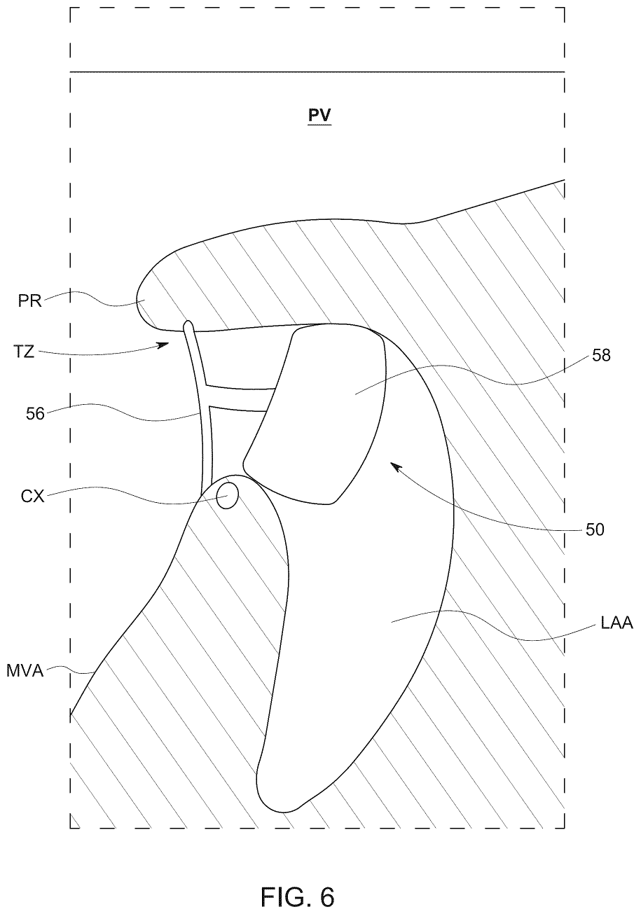

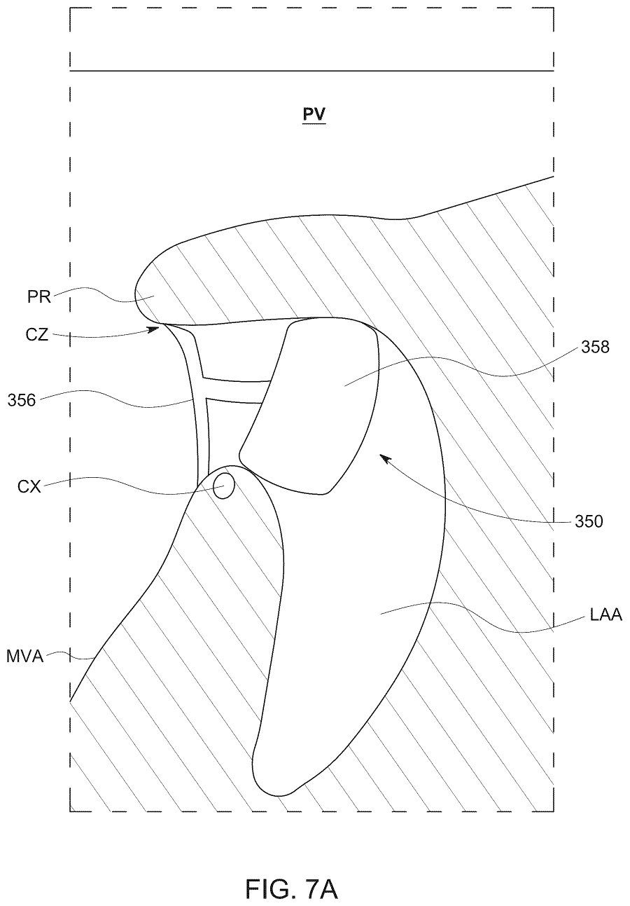

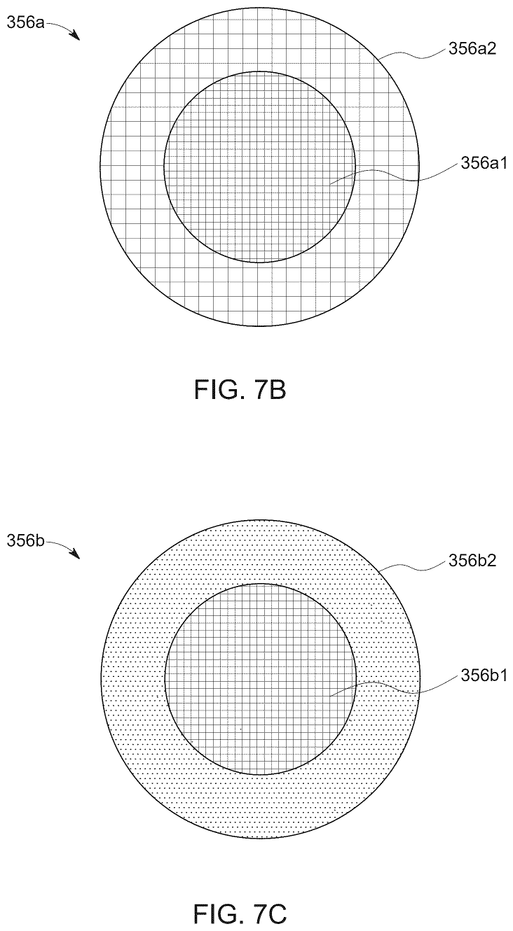

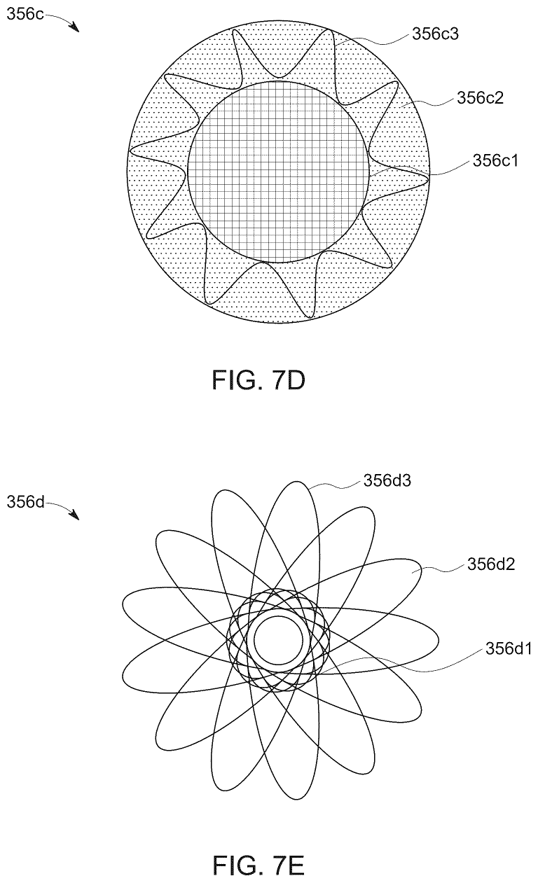

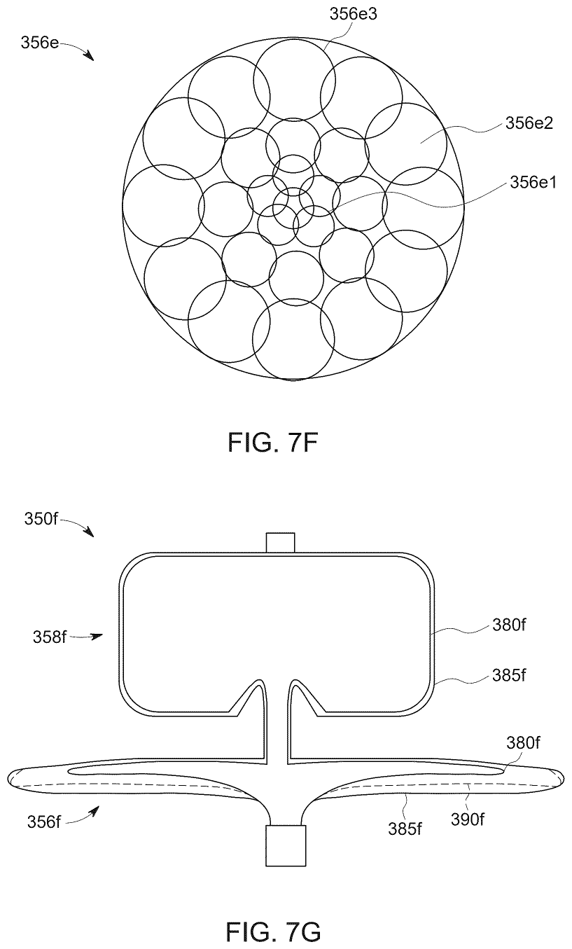

FIG. 6 is a highly schematic view of a version of medical device 250 of FIG. 1 implanted within an exemplary LAA. In the example of FIG. 6, the disc 56 has a relatively high stiffness. Stiffness of the disc 56 may be desirable to ensure that the disc 56 maintains a desired shape and so that the disc 56 is adequately supported. However, in some instances, similar to that shown in FIG. 6, the stiffness of the disc 56 may cause an edge of the disc 56 to press into tissue to cause the tissue to tent, indicated by tent zone TZ. This tent zone TZ may be another form of a stagnation zone SZ shown in FIG. 3, and it may be desirable to eliminate the tenting of the tissue while maintaining the structural benefits of maintaining stiffness of the disc 56. One way to achieve this is to provide for variable stiffness zones within the disc of the occluder. For example, FIG. 7A illustrates an occluder 350 implanted within the LAA, the occluder 350 including a lobe 358 and a disc 356. The occluder 350 may be identical to medical device 50, with one exception being that disc 356 may have variable stiffness, such that an outer zone (e.g., an outer radius) of the disc 356 has a relatively low stiffness while an inner zone (e.g., an inner radius) of the disc 356 has a relatively high stiffness. With this configuration, the stiff inner zone may provide support to the occluder 350 generally while helping the disc 356 maintain its general shape and/or form, and the relatively soft or compliant or flexible outer zone may provide enhanced flexibility and sealing against the tissue. For example, in the example shown in FIG. 7A, the outer zone of the disc 356 is more flexible and more able to bend and conform to the pulmonary ridge PR. Comparing FIG. 6 and FIG. 7A, it can be seen that the relatively stiff outer zone of disc 56 of the medical device 50 of FIG. 6 may result in the tent zone TZ described above, whereas the relatively flexible or soft outer zone of disc 356 of FIG. 7A may result in a conformability zone CZ. This conformability zone may result in the outer edge of the disc 356 conforming to the shape of the pulmonary ridge PR which it contacts to provide for a smooth transition between the ostium of the LAA and the pulmonary ridge PR, thus reducing or eliminating any stagnation zones (such as the tent zone TZ shown in FIG. 6 or the stagnation zone SZ shown in FIG. 3). There are various suitable ways in which the disc 356 may be provided with variable stiffness, including options described in U.S. Patent Application Publication No. 2021/0059684, the disclosure of which is hereby incorporated by reference herein.

In some examples, a softer or less stiff radially outer zone of disc 356 may be achieved by varying the thickness of the wires or strands forming the disc 356 by removal of material. The occluder 350 may include a tubular member comprising the proximal disc 356, the distal lobe 358, and a narrow connecting portion connecting the two, wherein the tubular member has an expanded configuration when deployed at the target site (e.g., the LAA) and a reduced configuration for delivery to the target site. The occluder 350 may be formed of at least one braided layer with material removed from a portion thereof, wherein the portion of the braided layer with material removed comprises a smaller braid wire diameter at outer radial portion(s) of the disc 356 compared to the inner radial portion(s) of the disc 356, as well as of the lobe 358 and/or connecting portion. This configuration may increase the compliance of the disc 356 at or near the contact points with tissue, while maintaining a structural strength of the remaining portions of the occluder 350. In some embodiments, the material may be removed from the wires or strands forming the outer radial portion(s) of the disc 356 by polishing (e.g., electropolishing or mechanically polishing) the outer radial portion(s) of the disc 356, without polishing the remaining areas of the occluder, so as to create a lower braid wire diameter in a localized region of the disc 356 at the outer radial zones while maintaining the larger wire diameter on the other portions of the occluder 350. In some embodiments, varying the braid wire thickness through targeted material removal (e.g., microblasting, acid, electropolishing, or some combination thereof) reduces the forces exerted by the outer edge(s) of the disc 356 while maintaining strength of other parts of the device (e.g., the radial force of the lobe 358, and allowing the center of the disc to maintain tension against the ostium to maintain sealing force). The amount of material removal depends on the desired reduction of force exerted by the outer edge of the disc 356. For example, if each braid wire starts at a diameter of about 0.007 inches (about 0.178 mm), removing material from the outer edge of disc 356 until the wire diameter is about 0.002 inches (about 0.051 mm) significantly reduces the force exerted on the anatomy after implanting the device occluder 350. However, in some embodiments, the braid wires may have a starting or nominal diameter of between about 0.003 inches (about 0.0762 mm) and about 0.005 inches (about 0.127 mm), and an amount of material may be removed so that an average reduction of wire diameter of between about 0.0005 inches (about 0.0127 mm) and about 0.002 inches (0.0508 mm) is achieved. In some embodiments, the material removal may be discrete so that there are only two zones with substantially similar wire diameters within each zone, but in other embodiments, the material removal may be gradual so that the wire diameter decreases continuously along a distance between the radial center of the disc 356 and the outer edge of the disc 356.

Braided occluders are typically made with a braid diameter that closely matches a diameter of a largest portion of the occluder. In exemplary embodiments, braid diameter is defined by a maximum expanded braid diameter and is a function of a diameter of the braid mandrel on which the braid is formed as well as a pick rate (i.e., picks per inch, or PPI) of the braid. In braided materials, PPI describes the number of braid wire crossings per inch of material, in which a pick (sometimes also referred to as a ‘pic’) is a single crossing of braid wires. Braid wire size (i.e., wire diameter) is dependent on multiple factors, including the size of the device, the forces required to secure the device in the anatomy, the number of braid wires (e.g., PPI), the purpose of the braid layer (occlusion vs. embolization resistance), the location of the defect, etc. In some embodiments, suitable wire sizes for braids used to form braided occluders of the present disclosure are in the range of about 0.001 inches diameter (about 0.0254 mm) to about 0.005 inches diameter (about 0.127 mm) wire.

In some examples, rather than (or in addition to) polishing braid wires to selectively reduce sizes thereof, the mechanical properties of the occluder can be further tailored by changing the pick rate (i.e., PPI) in different areas of the braid. FIG. 7B illustrates an exemplary braid pattern of disc 356a (which may be the same as disc 356 of occluder 350). Specifically, the pick rate at a radially inner or center zone 356a1 of the braid of the disc 356a is higher than a pick rate at a radially outer or edge zone 356a2 of the braid pattern located toward the outer edges of the disc 356a. In some embodiments, the differential braid pick configuration shown in FIG. 7B may be achieved using chase wires. That is, additional wires (or chase wires) of the same or larger diameter of wires used in the braid may be braided in conjunction with any number of wires in the braid and then removed from (e.g., cut out of) the radially or outer edge zone 356a2. Chase wire ends may need to be secured to adjacent wires (such as those forming the radially inner or center zone 356a1) or formed/directed inward towards the center of the device to prevent any traumatic wire ends during delivery, deployment, and defect occlusion. In other embodiments, the differential braid pick configuration shown in FIG. 7B may be achieved using wire forms. Formed wires of any shape, size or number may be interwoven into the braid only at the radially inner or center zone 356a1 of the device. Alternatively, the additional wires may be formed separately and attached to the radially inner or center zone 356a1 of the device by suturing or laminating the wire form to the center portion of the disc 356a. In some embodiments, a braided occluder is made with a braid pattern that transitions to a higher PPI near the radially inner or center zone 356a1 and a lower PPI near the radially outer or edge zone 356a2, which in some examples may help to improve (e.g., increase) elongation in areas of the device where it may be desirable, including for example through the waist (e.g. connecting portion) of the device.

In some examples, rather than (or in addition to) polishing braid wires to selectively reduce sizes thereof, the mechanical properties of the occluder can be further tailored by incorporating a relatively soft polymer into the disc. FIG. 7C illustrates an exemplary disc 356b (which may be the same as disc 356 of occluder 350). Specifically, disc 356b may have a radially inner or center zone 356b1 which may include a strands of wire formed into a braid similar to other embodiments described herein, such as similar to radially inner or center zone 356a1. However, disc 356b may include a radially outer or edge zone 356b2 which is mostly or totally devoid of metal wires. Instead of forming the radially outer or edge zone 356b2 of metal wires, it may be formed of a relatively soft polymer material (relatively soft compared to the radially inner or center zone 356b1). In some examples, the radially outer or edge zone 356b2 may be generally annular in shape, with an inner circumference that is coupled to the outer circumference of the radially inner or center zone 356b1, for example via sutures or adhesives. In some examples, the radially outer or edge zone 356b2 may be formed as a fabric made of one or more of polyethylene, high density polyethylene (“HDPE”), polypropylene, polyester, steralloy, tecothane, chronoprene, polyethylene terephthalate (“PTE”), polytetrafluoroethylene (“PTFE”), expanded PTFE (“ePTFE”), polyether bock amides, nylon, polyolefins, or combinations thereof. In other examples, the radially outer or edge zone 356b2 may be formed of a sheet (e.g. a solid piece of) one or more of the polymers described above. In other embodiments, the radially outer or edge zone 356b2 may be formed of a tissue instead of (or in addition to) a polymer, such as pericardial tissue, including porcine, bovine, or equine pericardial tissue, or collagen matrices. In other embodiments, the radially outer or edge zone 356b2 may be formed of a bioabsorbable polymer, including for example poly-L-lactic acid (“PLLA”), poly(glycolic acid) (“PGA”), copolyesters of poly(e-caprolactone) (“PCL”), poly(lactic-co-glycolic acid) (“PLGA”), poly(D,L-lactide-co-glycolide) (“PDLGA”), poly(L-co-D,L lactic acid) (“PLDLA”), olycaprolactone (“PCL”), trimethylene carbonate (“TMC”), poly(d-diozanone) (“PPDO”), and combinations of various polymers. With this configuration, the radially inner or center zone 356b1 may retain a desirable level of stiffness for structural support, for example so that tension on the disc 356b caused by the lobe (and/or the connection portion connecting the lobe to the disc 356b) is able to pull the disc 356b into good sealing contact with the ostium of the LAA. However, as should be understood, the relative softness or conformability of the radially outer or edge zone 356b2 may allow the disc 356b to better conform to the pulmonary ridge PR while avoiding a tenting zone TZ or otherwise minimizing or avoiding any stagnation zones SZ.

Although the example disc 356b of FIG. 7C in some examples is formed from polymer while being devoid of metal wires, in other embodiments some amount of wire support may be provided. For example, FIG. 7D illustrates an exemplary disc 356c (which may be the same as disc 356 of occluder 350). Specifically, disc 356c may have a radially inner or center zone 356c1 which may include a strands of wire formed into a braid similar to other embodiments described herein, such as similar to radially inner or center zone 356a1 or 356b1. However, while disc 356c may include a radially outer or edge zone 356c2 which is formed of a relatively soft polymer (similar or identical to radially outer or edge zone 356b2), the radially outer or edge zone 356c2 may also include one or more support wires 356c3. In the example of FIG. 7D, a single undulating support wire 356c3, which may be formed of nitinol or another shape memory material, and which may be the same type of wire strand to form the braid of the radially inner or center zone 356cl, may extend around the radially outer or edge zone 356c2. In this example, the undulating support wire 356c3 may extend from (or near) the radial outer edge of the center zone 356cl to the radial outer edge of the edge zone 356c2, back toward the radial outer edge of the center zone 356c1, and so on. With this configuration, the support wire 356c3 undulates in a sinusoidal fashion around the circumference of the radially outer or edge zone 356c2. The support wire 356c3 may provide extra support to the relatively soft polymer, without being so stiff as to change the general relationship in which the radially outer or edge zone 356c2 is softer and/or more compliant than the radially inner or center zone 356c1. Although the specific embodiment of FIG. 7D shows a single support wire 356c3, it should be understood that more than one support wire may be provided, either as individual or braided wires, preferably with the entirety of the radially outer or edge zone 356c2 maintaining, in total, a higher softness (smaller/lower stiffness) and/or higher conformability than the radially inner or center zone 356c1. In some examples, the polymer of the radially outer edge zone 356c2 may be coupled to the support wire 356c3 via sutures, adhesives, or any other suitable means. In some embodiments, the polymer may be laminated over the support wire 356c3 to encapsulate the support wire 356c3 therein. In some embodiments, a single layer of the polymer may be provided, or two (or more) layers of the polymer may be provided so as to sandwich the support wire 356c3 between two or more of the layers of polymer. Although support wire 356c3 is shown as a single undulating member, it should be understood that other similar alternative constructions may be suitable, such as having a plurality of individual support wires extend radially outwardly from the radial outer edge of the center zone 356cl to the radially outer edge of the radially outer or edge zone 356c2, similar to spokes of a bicycle wheel.

In some examples, rather than (or in addition to) polishing braid wires to selectively reduce sizes thereof and/or incorporating soft polymers into the disc, the mechanical properties of the occluder can be further tailored by providing particular geometrical shapes of the wire forming the disc. FIG. 7E illustrates an exemplary disc 356d (which may be the same as disc 356 of occluder 350). Specifically, disc 356d may have a radially inner or center zone 356d1 which is relatively stiff compared to a radially outer or edge zone 356d2 which is relatively soft and/or conformable. Rather than the disc 356d being formed of a relatively consistent braid with relatively consistent properties, the disc 356d of FIG. 7E is formed with a wire (which may be a nitinol wire or other shape memory material) that forms a plurality of loops or spindles 356d3. Each spindle 356d3 may have a generally oval or circular shape, although other shapes may be suitable. In some examples, a single wire may form all of the spindles 356d3, although in other embodiments each spindle 356d3 may be formed of its own wire, and in some examples the spindles 356d3 may each be formed of multiple wires. Preferably, at least in part because of the geometry of the spindles 356d3 relative to the disc 356d, there is more wire material (e.g. a greater density of wire material) near a center point where the spindles 356d3 all meet (or at least are all close to each other if they do not actually meet each other) and the density of wire material decreases in a direction radially outward from the center point toward the outer radial edge of the disc 356d. With this configuration, the radially inner or center zone 356d1 has a higher stiffness (or smaller softness) than the radially outer or edge zone 356d2 due to the density of material decreasing in the direction toward the outer radial edge. It should be understood that this configuration may generally create a gradient in which the stiffness gradually decreases (or in some cases in which the stiffness has a step-wise decrease) from the radial center of the disc 356d toward the outer radial edge of the disc 356d. It should be understood that, as with other embodiments of disc 356, disc 356d may include an occluding fabric on and/or within disc 356d. Additional details regarding similar discs are described in U.S. Patent Application Publication No. 2022/0395266, the disclosure of which is hereby incorporated by reference herein.

The wire geometry of disc 356d of FIG. 7E is not the only way in which the disc can be formed with a relatively soft or conformable outer edge. For example, FIG. 7F illustrates an alternate embodiment of the disc 356d of FIG. 7E. In particular, FIG. 7F illustrates an embodiment of disc 356e in which the disc 356e is formed of a plurality of wire loops 356e3 of differing sizes. The wire loops 356e3 may each be formed of a single wire or multiple wires, and in some embodiments, all of the wire loops 356e3 may be formed of a single wire. The wire loops 356e3 have smaller sizes (e.g. smaller internal area) near the radial center of the disc 356e, and the wire loops 356e3 increase in size in a direction toward the outer periphery of the disc 356e. With this configuration, similar to disc 356d, there is more wire material (e.g., a greater density of material) closer to the radial center of the disc 356e, and less wire material (e.g., a lower density of material) toward the outer periphery of the disc 356e. This may result in a radially inner or center zone 356e1 that is relatively stiff or relatively non-conformable compared to a radially outer or edge zone 356e2.

In another embodiment, an example of which is shown in FIG. 7G, occluder 350f may be provided with two braid layers. In the example of FIG. 7G, occluder 350f includes a lobe 358f and a disc 356f similar to other occluders described herein. However, unlike other occluders described herein, occluder 350f may be formed of two layers of braided material (e.g. two separate layers of mesh formed by braiding strands of nitinol together), including an inner braid layer 380f and an outer braid layer 385f. In some examples, the inner braid layer 380f may be formed with higher stiffness (e.g., less conformability) than the outer braid layer 385f. In some examples, the inner braid layer 380f may be positioned in close proximity to the outer braid layer 385f in the area of the lobe 358f, while in the area of the disc 356f, the outer edge of the inner braid layer 380f is a larger spaced distance away from the outer edge of the outer braid layer 385f. Similar to other embodiments described herein, this configuration may allow for the outer radial portions of the disc 356f to have more softness or greater conformability since only (or mostly only) the outer braid layer 385f, which is relatively soft, may interact with tissue upon implantation, while the stiffer inner braid layer 380f is capable of providing the desirable structural support to the occluder 350f. Also shown in FIG. 7G is an occlusive fabric 390f. If the occlusive fabric 390f is included in the disc 356f, it may be included between the inner braid layer 380f and the outer braid layer 385f, although other configurations are suitable. Additional details regarding forming a disc with a double-layer braid are described in U.S. Patent Application Publication No. 2023/0404559, the disclosure of which is hereby incorporated by reference herein.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A collapsible and expandable medical device for occluding a left atrial appendage (“LAA”), the medical device comprising:
a proximal disc configured to cover an ostium of the LAA in an implanted condition of the medical device;
a distal lobe configured to be received within a cavity of the LAA in the implanted condition of the medical device, the distal lobe including a central longitudinal axis extending therethrough in an expanded condition of the medical device; and
a connecting member connecting the proximal disc to the distal lobe;
wherein in the expanded condition of the medical device, the proximal disc is not radially symmetric about the central longitudinal axis of the distal lobe.

2. The medical device of claim 1, wherein in the expanded condition of the medical device, the connecting member extends along a central longitudinal axis that is oblique to the central longitudinal axis of the distal lobe.

3. The medical device of claim 1, wherein in the expanded condition of the medical device, the proximal disc extends along a central longitudinal axis, the central longitudinal axis of the proximal disc being offset and parallel to the central longitudinal axis of the distal lobe.

4. The medical device of claim 3, wherein the distal lobe is radially symmetric about the central longitudinal axis of the distal lobe, and the proximal disc is radially symmetric about the longitudinal axis of the proximal disc.

5. The medical device of claim 3, wherein in the expanded condition of the medical device, the central longitudinal axis of the proximal disc passes through the distal lobe.

6. The medical device of claim 5, further comprising at least one radiopaque marker coupled to a radially outer surface of the distal lobe at a position of the radially outer surface of the distal lobe having a minimum spaced distance radially from the central longitudinal axis of the proximal disc.

7. The medical device of claim 5, further comprising at least one radiopaque marker coupled to a radially outer surface of the proximal disc at a position of the radially outer surface of the proximal disc having a maximum spaced distance radially from the central longitudinal axis of the distal lobe.

8. The medical device of claim 5, further comprising at least one radiopaque marker coupled to a radially outer surface of the distal lobe at a position of the radially outer surface of the distal lobe having a maximum spaced distance radially from the central longitudinal axis of the proximal disc.

9. The medical device of claim 1, wherein in the expanded condition of the medical device, the proximal disc extends along a central longitudinal axis, and the proximal disc is not radially symmetric about the central longitudinal axis of the proximal disc.

10. The medical device of claim 1, wherein in the expanded condition of the medical device, the proximal disc includes an outer periphery with a first portion and a second portion that together form the outer periphery, the first portion having a first radius of curvature, the second portion having a second radius of curvature larger than the first radius of curvature.

11. The medical device of claim 10, wherein the first portion of the outer periphery of the proximal disc forms an arc having a center that is substantially coaxial with the central longitudinal axis of the distal lobe.

12. The medical device of claim 11, wherein the second portion of the outer periphery of the proximal disc forms an arc having a center that is not coaxial with the central longitudinal axis of the distal lobe.

13. A collapsible and expandable medical device for occluding a left atrial appendage (“LAA”), the medical device comprising:
a proximal disc configured to cover an ostium of the LAA in an implanted condition of the medical device;
a distal lobe configured to be received within a cavity of the LAA in the implanted condition of the medical device, the distal lobe including a central longitudinal axis extending therethrough in an expanded condition of the medical device; and
a connecting member connecting the proximal disc to the distal lobe;
wherein the proximal disc includes a radially outer zone forming an outer periphery of the proximal disc and a radially inner zone that is positioned radially inwards of the radially outer zone, the radially inner zone having a stiffness that is greater than a stiffness of the radially outer zone.

14. The medical device of claim 13, wherein the proximal disc is formed of one or more strands of wires braided together, the one or more strands located in the radially outer zone having a thickness that is smaller than a thickness of the one or more strands located in the radially inner zone.

15. The medical device of claim 13, wherein the proximal disc is formed of one or more strands of wires braided together into a braided fabric with each braid wire crossing forming a pick, a pick rate being defined as the number of picks per inch of braided fabric, the radially outer zone having a pick rate that is smaller than a pick rate of the radially inner zone.

16. The medical device of claim 13, wherein the radially inner zone of the proximal disc is formed of one or more strands of wires braided together, and the radially outer zone of the proximal disc includes a polymer.

17. The medical device of claim 16, wherein the radially outer zone of the proximal disc is devoid of wire strands.

18. The medical device of claim 16, wherein the radially outer zone of the proximal disc includes at least one wire strand.

19. The medical device of claim 18, wherein the at least one wire strand in the radially outer zone of the proximal disc undulates between a radially outer edge of the radially inner zone and a radially outer edge of the radially outer zone in a circumferential direction of the radially outer zone.

20. The medical device of claim 13, wherein the proximal disc is formed by one or more wire strands that form a plurality of spindles extending from a central portion of the proximal disc toward a radially outer edge of the proximal disc such that the radially inner zone has a density of wire strands that is greater than a density of wire strands in the radially outer zone.

21. The medical device of claim 13, wherein the proximal disc is formed by one or more wire strands that form a plurality of wire loops, a first group of wire loops being positioned relatively close to a radial center of the proximal disc each having a first size, a second group of wire loops being positioned relatively close to an outer peripheral edge of the proximal disc each having a second size, the first size being smaller than the second size such that the radially inner zone has a density of wire strands that is greater than a density of wire strands in the radially outer zone.
